(19) United States
(12) Patent Application Publication
(10) Pub. No.: US 2024/0010672 A1
(43) Pub. Date: Jan. 11, 2024

(54) MATERIALS AND METHODS FOR PROTEIN PROCESSING

(71) Applicant: AMGEN INC., Thousand Oaks, CA (US)

(72) Inventors: Sreekanth SURAVAJJALA, Thousand Oaks, CA (US); Jennifer Lynn LIPPENS, Thousand Oaks, CA (US)

(21) Appl. No.: 18/251,553

(22) PCT Filed: Nov. 1, 2021
(86) PCT No.: PCT/US21/57519

§ 371 Date: May 3, 2023

Related U.S. Application Data
(60) Provisional application No. 63/110,087, filed on Nov. 5, 2020.

(51) Int. Cl. C07K 1/107; C07K 1/16; C07K 16/46

(52) U.S. Cl. CPC C07K 1/107; C07K 1/16; C07K 16/468; C07K 2317/565; C07K 2317/31

(57) ABSTRACT

Described herein are materials and methods for protein production and analysis.

### FIELD

The presented subject matter relaters to the field of polypeptide analysis.

### INCORPORATION BY REFERENCE FOR MATERIAL SUBMITTED ELECTRONICALLY

The present application is being filed with a sequence listing in electronic format. The sequence listing provided as a file titled, “55406_Seqlisting.txt,” created Oct. 13, 2021 and is 268,106 bytes in size. The information in the electronic format of the sequence listing is incorporated herein by reference in its entirety.

### BACKGROUND

Peptide mapping is a valuable approach to combine positional quantitative information with topographical and domain information of proteins. In particular, annotated peptide mapping is a useful procedure and a critical goal of many biomedical and biopharmaceutical research and production efforts.

Proteins are complex large molecules and biological production and characterization of protein pharmaceuticals (“biologics”) poses many demanding analytical challenges that do not arise from small molecule drugs. Biologics are prone to production challenges such as sequence variation, misfolding, variant glycosylation, and post-translational degradation including aggregation and modifications such as oxidation and deamidation. These challenges can lead to loss of safety and efficacy, so there is a need in the biopharmaceutical industry to identify and quantify variant and degraded forms of the product down to low concentrations, plus obtain tertiary structure information.

### SUMMARY

In one aspect, described herein is a method of processing a protein comprising fragmenting the protein under enzymatic and/or non-enzymatic conditions to generate polypeptides to produce polypeptides; applying polypeptides to a chromatography column; and eluting the polypeptides in an eluant comprising a mobile phase B solvent, wherein the mobile phase B solvent comprises trifluoroacetic acid (TFA); acetonitrile; and alcohol. In some embodiments, the protein comprises a complementarity determining region (CDR) of a variable region of an antigen binding protein. For example, the protein may comprise a CDR3 of a heavy chain variable region (HCDR3) and/or a CDR3 of a light chain variable region (LCDR3), and the method further comprises constructing a structural map of the protein, wherein the structural map comprises the HCDR3 and the LCDR3. In some embodiments, the mobile phase B comprises 0.05%-0.09% TFA.

In some embodiments, at least 50% of the HCDR3 containing polypeptide and/or at least 50% of the LCDR3 containing polypeptide is eluted from the chromatography column. In some embodiments, the mobile phase B comprises the TFA in about 35-45% acetonitrile, 35-45% alcohol, and water. Exemplary alcohols include, but are not limited to, isopropyl alcohol, propanol and butyl alcohol.

In some embodiments, the eluting step is carried out on a gradient of the mobile phase B solvent and a polar mobile A solvent. In some embodiments, the mobile phase A comprises TFA and water. In some embodiments, the mobile phase A comprises less than 0.1% TFA (e.g., 0.05%, 0.06%, 0.07%, 0.08% or 0.09% TFA).

Chromatography columns for use in accordance with the methods described herein include columns comprising porous particles having a particle size of about 2 μm to about 7 μm (e.g., 2 μm, 3 μm, 4 μm, 5 μm, 6 μm or 7 μm, including ranges between any two of the listed values). In some embodiments, the chromatography column comprises porous particles each having a pore size of about 100-500 angstroms (e.g., about 100, about 200, about 300, about 400, or about 500 angstroms). In some embodiments, the porous particles each have a pore size of about 300 angstroms and a particle size of about 3 μm. In some embodiments, the chromatography column comprises a divinylbenzene (DVB) resin.

The chromatography columns for use in the methods described herein may be at least 10 cm (e.g., at least 10 cm, at least 15 cm, at least 20 cm, at least 25 cm, or at least 30 cm) in height.

In some embodiments, the methods described herein further comprises performing spectrometric analysis of the eluted polypeptides.

In some embodiments, the protein comprises a therapeutic protein. Therapeutic proteins include, but are not limited to, an antibody or antigen-binding fragment thereof, a derivative of an antibody or antibody fragment, or a fusion polypeptide. In some embodiments, the therapeutic protein is infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, and zolimomab aritox. In yet other sub-aspects, the therapeutic polypeptide is a polypeptide selected from the group consisting a glycoprotein, CD polypeptide, a HER receptor polypeptide, a cell adhesion polypeptide, a growth factor polypeptide, an insulin polypeptide, an insulin-related polypeptide, a coagulation polypeptide, a coagulation-related polypeptide, albumin, IgE, a blood group antigen, a colony stimulating factor, a receptor, a neurotrophic factor, an interferon, an interleukin, a viral antigen, a lipoprotein, calcitonin, glucagon, atrial natriuretic factor, lung surfactant, tumor necrosis factor-alpha and -beta, enkephalinase, mouse gonadotropin-associated peptide, DNAse, inhibin, activing, an integrin, protein A, protein D, a rheumatoid factor, an immunotoxin, a bone morphogenetic protein, a superoxide dismutase, a surface membrane polypeptide, a decay accelerating factor, an HIV envelope, a transport polypeptide, a homing receptor, an addressin, a regulatory polypeptide, an immunoadhesin, a myostatin, a TALL polypeptide, an amyloid polypeptide, a thymic stromal lymphopoietin, a RANK ligand, a c-kit polypeptide, a TNF receptor, or an angiopoietin, and biologically active fragments, analogs or variants thereof.

In some embodiments, the therapeutic protein comprises a BiTE® (bi-specific T-cell engager) molecule. For example, the therapeutic protein may comprise a half-life extended (HLE) BiTE® molecule.

In another aspect, disclosed herein is a chromatography column comprising polypeptide fragments of a protein; and an eluant comprising a mobile phase B solvent comprising: trifluoroacetic acid (TFA); acetonitrile; and alcohol. In some embodiments, the protein comprises a CDR of a variable region. In some embodiments, the protein comprises a CDR3 of a heavy chain variable region and/or a CDR3 of a light chain variable region. In some embodiments, the eluant comprises a greater quantity of the HCDR3 than is bound to the column, and/or a greater quantity of the LCDR3 than is bound to the column.

In some embodiments, the mobile phase B on any of the disclosed chromatography columns comprises TFA in about 35-45% acetonitrile, 35-45% alcohol, and water. In some embodiments, the mobile phase B comprises TFA in about 40% acetonitrile, 40% alcohol, and 20% water. Exemplary alcohols include, but are not limited to, isopropyl alcohol, propanol and butyl alcohol.

In some embodiments, the eluant of the chromatography column is a gradient of the mobile phase B solvent and a polar mobile A solvent. In some embodiments, the mobile phase comprises TFA and water. In some embodiments, the mobile phase A comprises less than 0.1% TFA (e.g., 0.05%, 0.06%, 0.07%, 0.08% or 0.09% TFA).

In some embodiments, the chromatography column comprises porous particles having a particle size of about 2 μm to about 7 μm (e.g., 2 μm, 3 μm, 4 μm, 5 μm, 6 μm or 7 μm, including ranges between any two of the listed values). In some embodiments, the chromatography column comprises porous particles each having a pore size of about 100-500 angstroms (e.g., about 100, about 200, about 300, about 400, or about 500 angstroms). In some embodiments, the porous particles each have a pore size of about 300 angstroms. In some embodiments, the chromatography column comprises fully porous particles having a pore size of about 300 angstroms and a particle size of about 5 μm.

Any of the disclosed chromatography columns are at least 10 cm (e.g., at least 10 cm, at least 15 cm, at least 20 cm, at least 25 cm, or at least 30 cm) in height.

The use of the singular includes the plural unless specifically stated otherwise. The use of “or” means “and/or” unless stated otherwise. The use of the term “including”, as well as other forms, such as “includes” and “included,” is not limiting. Terms such as “element” or “component” encompass both elements and components comprising one unit and elements and components that comprise more than one subunit unless specifically stated otherwise. The use of the term “portion” can include part of a moiety or the entire moiety. When a numerical range is mentioned, e.g., 1-5, all intervening values are explicitly included, such as 1, 2, 3, 4, and 5, as well as fractions thereof, such as 1.5, 2.2, 3.4, and 4.1.

“About” or “˜” means, when modifying a quantity (e.g., “about” 3 mM), that variation around the modified quantity can occur. These variations can occur by a variety of means, such as typical measuring and handling procedures, inadvertent errors, ingredient purity, and the like.

“Comprising” and “comprises” are intended to mean that methods include the listed elements but do not exclude other unlisted elements. The terms “consisting essentially of” and “consists essentially of,” when used in the disclosed methods include the listed elements, exclude unlisted elements that alter the basic nature of the method, but do not exclude other unlisted elements. The terms “consisting of” and “consists of” when used to define methods exclude substantial method steps. Embodiments defined by each of these transition terms are within the scope of this disclosure.

### DETAILED DESCRIPTION

Peptide mapping is widely used in the biopharmaceutical industry and its applications range from advanced characterization methods to key routine release multi-attribute testing assays which replace several conventional methods in release specifications.

The CDR regions of monoclonal antibodies (such as HCDR1, HCDR2, HCDR3, LCDR1, LCDR2, and LCDR3) are known to impact the overall drug efficacy through antigen-antibody mediated binding properties, Any post-translational modifications (PTMs) in this region need to be carefully assessed for their impact on efficacy and safety of the product. As described herein in the Examples, the recovery of CDR-containing peptides from several therapeutic proteins was examined in peptide mapping assays using conventional column chemistries. The findings indicated that recoveries of some CDR-containing polypeptides using conventional column chemistries were poor, and as a result identification and quantitation of post-translational modifications (PTMs) on these CDR-containing peptides was challenging. Described herein is a peptide mapping method that can be used under both reduced and non-reduced conditions which improves recovery of these peptides and significantly improves the quantitation and identification of previously undetermined PTMs. PTMs include, but are not limited to, site-specific glycosylation, isomers, covalent bonds, oxidation, deamidation, hydroxylation, glycation, amino acid substitutions (sequence variants) and/or truncations.

In one aspect, described herein is a method of processing a protein comprising fragmenting the protein to produce polypeptides; applying polypeptides to a chromatography column; and eluting the polypeptides in an eluant comprising a mobile phase B solvent comprising trifluoroacetic acid (TFA); acetonitrile; and alcohol. In some embodiments, the protein is reduced. The term “reduced protein” (and similar terms) as used herein means a protein in which at least one of its interchain or intrachain disulfide bonds is broken. Such disulfide bonds can form between reduced thiol groups, such as those available on cysteine residues. In other embodiments, the protein is non-reduced. The term “non-reduced protein” (and similar terms) as used herein means a protein in which at least one of its interchain or intrachain disulfide bonds are intact. In some embodiments, the method comprises reducing the protein.

The methods disclosed herein may comprise fragmenting the protein to be analyzed in a sample, wherein the fragmentation produces at least two polypeptide fragments of the protein. Any suitable method of fragmenting the protein can be used, provided that at least two polypeptide fragments of the protein are produced. For example, the protein may be cleaved by a protease or chemical, and/or fragmented by thermal degradation. In some embodiments, at least three polypeptide fragments of the protein are produced. In some embodiments, at least four, five, six, seven, eight, nine or ten fragments are produced.

In some embodiments, the protein is cleaved by a protease. Any suitable protease can be used, as long as such protease cleaves the protein into at least two polypeptide fragments. Exemplary proteases include, but are not limited to, trypsin, neutrophil elastase. endoproteinase Glu-C, endoproteinase Arg-C, pepsin, chymotrypsin, chymotrypsin B, Lys-N protease, Lys-C protease, Glu-C protease, Asp-N protease, pancreatopeptidase, carboxypeptidase A, carboxypeptidase B, proteinase K, and thermolysin. In some embodiments, the protein is cleaved by two or more proteases.

In some embodiments, the protein and protease are combined at protein: protease ratio (w/w) of 10:1, 20:1, 25:1, 50:1, or 100:1. In some embodiments, the ratio is 20:1. In some embodiments, the protease used is at a concentration of about 100 ng/ml-1 mg/ml, or about 100 ng/ml-500 μg/ml, or about 100 ng/ml-100 μg/ml, or about 1 μg/ml-1 mg/ml, or about 1 μg/ml-500 μg/ml, or about 1 μg/ml-100 μg/ml, or about 10 μg/mg-1 mg/ml, or about 10 μg/mg-500 μg/ml, or about 10 μg/mg-100 μg/ml. In some embodiments, the fragmenting step is performed for about 10 minutes to about 48 hours, or about 30 minutes to about 48 hours, or about 30 minutes to about 24 hours, or about 30 minutes to about 16 hours, or about 1 hour to about 48 hours, or about 1 hour to about 24 hours, or about 1 hour to about 16 hours, or about 1 to about 8 hours, or about 1 to about 6 hours, or about 1 to about 4 hours. In some embodiments, the fragmenting step is performed at a temperature between about 20° C. and about 45° C., or between about 20° C. and about 40° C., or between about 22° C. and about 40° C., or between about 25° C. and about 37° C. In some embodiments, the fragmenting step is performed at about 37° C. One of skill in the art can choose appropriate conditions (buffers, incubation times, amount of protease, volumes, etc.), as in vitro protease digestion is understood in the art.

In some embodiments, the fragmenting of the protein into polypeptide fragments is accomplished using a chemical, preferably a chemical that cleaves a protein in a site-specific manner. Such chemicals include cyanogen bromide (CNBr; carbononitridic bromide), which cleaves C-terminal of methionine residues; 2-nitro-5-thiocyanobenzoate (NTCB), which cleaves N-terminally of cysteine residues; asparagine-glycine dipeptides can be cleaved using hydroxlamine; formic acid, which cleaves at aspartic acid-proline (Asp-Pro) peptide bonds, and BNPS-skatole (3-bromo-3-methyl-2-(2-nitrophenyl)sulfanylindole), which cleaves C-terminal of tryptophan residues. One of skill in the art understands how to select appropriate variables, including polypeptide concentration, chemical concentration, incubation time and temperature, etc. See also, for example, (Crimmins et al., 2001; Li et al., 2001; Tanabe et al., 2014).

Chromatographic Methods

Chromatographic methods are those methods that separate polypeptide fragments in a mobile phase, which phase is processed through a structure holding a stationary phase. For example, a chromatography column is a structure holding a stationary phase during chromatography. Because the polypeptide fragments are of different sizes and compositions, each fragment has its own partition coefficient. Because of the different partition coefficients, the polypeptides are differentially retained on the stationary phase. Exemplary chromatography methods include, but are not limited to, include gas chromatography, liquid chromatography, high performance liquid chromatography, ultra-performance liquid chromatography, size-exclusion chromatography, ion-exchange chromatography, affinity chromatography, expanded bed adsorption chromatography, reverse-phase chromatography, and hydrophobic interaction chromatography.

The chromatography methods described herein comprise the step of applying polypeptides to a chromatography column and eluting the polypeptides in an eluant comprising a mobile phase B solvent comprising trifluoroacetic acid (TFA), acetonitrile and alcohol.

In some embodiments, the mobile phase B comprises TFA in about 30% acetonitrile (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, or 35-45%). The mobile phase B may comprise less than 0.1% TFA (e.g., 0.09%, 0.07%, 0.06%, 0.05% TFA). In some embodiments, the mobile phase B comprises TFA in about 30% alcohol (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, 35-40%, or 35-45%). The mobile phase B may comprise less than 0.1% TFA (e.g., 0.09%, 0.08%, 0.07%, 0.06%, 0.05% TFA).

In some embodiments, the mobile phase B comprises TFA in about 30% acetonitrile (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, or 35-45%); about 30% alcohol (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, 35-40%, or 35-45%); and water. The mobile phase B may comprise less than 0.1% TFA (e.g., 0.09%, 0.08%, 0.07%, 0.06%, 0.05% TFA). In some embodiments, the mobile phase B comprises TFA in about 30% acetonitrile (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, 35-40%, or 35-45%); and about 35-45% alcohol; and water. In some embodiments, the mobile phase B comprises TFA in about 35-45% acetonitrile; about 30% alcohol (e.g., about 31%, about 32%, about 33%, about 34%, about 35%, about 36%, about 37%, about 39%, about 40%, about 41%, about 42%, about 43%, about 44%, or about 45%, including ranges between any two of the listed values, for example 30-40%, 30-45%, 35-40%, or 35-45%); and water. The mobile phase B may comprise less than 0.1% TFA (e.g., 0.09%, 0.08%, 0.07%, 0.06%, 0.05% TFA).

In some embodiments, the mobile phase B comprises TFA in about 30% acetonitrile, about 30% alcohol, and water. In some embodiments, the mobile phase B comprises TFA in about 35% acetonitrile, about 35% alcohol, and water. In some embodiments, the mobile phase B comprises TFA in about 35-45% acetonitrile, about 35-45% alcohol, and water. In some embodiments, the mobile phase B comprises less than 0.1% TFA (e.g., 0.09%, 0.08%, 0.07%, 0.06%, 0.05% TFA). In some embodiments, the mobile phase B comprises about 0.05%-0.09% TFA. In some embodiments, the mobile phase B comprises 40% isopropanol, 40% acetonitrile, 20% water and 0.05% TFA.

Exemplary alcohols in the mobile phase B include, but are not limited to isopropyl alcohol, propanol and butyl alcohol.

In some embodiments the eluting step is on a gradient of the mobile phase B and a polar mobile phase A solvent. In some embodiments, the mobile phase A comprises trifluoroacetic acid (TFA) and water. In some embodiments, the mobile phase A comprises less than 0.1% TFA (e.g., 0.09%, 0.08%, 0.07%, 0.06%, 0.05% TFA). In some embodiments, the mobile phase A comprises about 0.05%-0.09% TFA. In some embodiments, the mobile phase A is water and 0.05% TFA.

In some embodiments, the chromatography column used in the methods described herein is a polymer-based column or a silica-based column. In some embodiments, the chromatography column comprises a divinylbenzene (DVB) resin. In some embodiments, the chromatography column comprises a polystyrene and divinylbenzene (DVB) resin. An example of an available column comprising DVB resin is a PLPR-S column (AGILENT), though this column conventionally has not been recommended or used for peptide mapping applications. Another chromatography column option for methods described herein is a graphitized carbon column.

It is observed herein that pore size can impact peptide recovery, as conventional C8 and C18 columns with pore sizes of about 1.7 μm (Examples 1-2) yielded lower CDR3 peptide recovery than columns with larger pore sizes of 3-5 μm (Examples 3-4). In some embodiments, the chromatography column comprises porous particles having a particle size of at least 2 μm. In some embodiments, the chromatography column comprises porous particles having a particle size of at least about 2 μm, or about 3 μm, or about 4 μm, or about μm, or about 6 μm, or about 7 μm, or about 8 μm, or about 9 μm, or about 10 μm, including ranges between any two of the listed values. In some embodiments, the chromatography column comprises porous particles having a particle size of about 2-5 μm, about 2-7 μm, about 3-5 μm or about 3-7 μm.

In some embodiments, the chromatography column comprises porous particles such as fully porous particles or superficially porous particles having a pore size of at least about 100 angstroms (e.g., about 100, about 200, about 300, about 400 or about 500 angstroms, including ranges between any two of the listed values, for example 100-500 angstroms). In some embodiments, the chromatography column comprises fully porous particles having a pore size of 300 angstroms and a particle size of about 5 μm. The term “fully porous particle” as used herein refers to a particle having a porous core and a porous outer shell. The term “superficially porous particles” as used herein refers to a particle having a solid core and a porous outer shell.

In some embodiments, the protein is an antibody and comprises a CDR3 of a heavy chain variable region (HCDR3) and/or a CDR3 of the light chain variable region (LCDR3). The methods described herein can be used to identify the amount of unmodified H-CDR3-containing polypeptides and/or unmodified LCDR3-containing polypeptides eluted from the chromatography column. The term “unmodified” as used herein with reference to HCDR3-containing polypeptides and/or CDR3-containing polypeptides eluted from the chromatography column refers to HCDR3- or LCDR3-containing polypeptides without post-translational modifications (PTMs).

Advantageously, methods described herein can facilitate recovery of hydrophobic peptides such as CDR3 peptides (e.g., HCDR3 and/or LCDR3 peptides). Conventional peptide mapping methods may not recover sufficient HCDR3 and/or LCDR3 to permit peptide mapping of the HCDR3 and/or LCDR3 region (respectively). For example, when less than 10% of a peptide is recovered from the chromatography column, detection of that peptide may be limited, and quantification of modified peptides may be inaccurate. Conventional methods have yielded about 1% to 2% recovery of CDR3 peptides of some proteins. Accordingly, conventional peptide mapping may obtain coverage of no more than about 85%, 90%, or 95% of the light chain or heavy chain, and CDR3 regions may be absent from the coverage (See Examples 1-2). However, according to methods of some embodiments herein, at least 10%, 15%, 20%, 25%, or 30% of CDR3 peptides may be recovered. As such, methods described herein can recover HCDR3 and/or LCDR3 peptides to produce peptide maps comprising coverage of the HCDR3 and/or LCDR3 (respectively). The peptide map may cover all or substantially of the protein. For example, methods described herein may yield at least 96%, 97%, 98%, 99% or 99.5%, or 100% coverage of the protein. (See Examples 3-4). In some embodiments, the method yields coverage of at least 96%, 97%, 98%, 99% or 99.5% of the light chain polypeptide, the heavy chain polypeptide, or the light chain and heavy chain polypeptide. In some embodiments, the method yields coverage of 100% of the light chain polypeptide, the heavy chain polypeptide, or the light chain and heavy chain polypeptide.

In some embodiments, at least 50% of the HCDR3-containing polypeptides and/or at least 50% of the LCDR3-containing polypeptides eluted from the chromatography column are unmodified (e.g., lack post-translational modifications, PTMs). In some embodiments, at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% of the HCDR3-containing polypeptides is eluted from the chromatography column are unmodified. In some embodiments, at least 50%, 55%, 60%, 65%, 70%, 75%, 80%, 85%, 86%, 87%, 88%, 89%, 90%, 91%, 92%, 93%, 94%, 95%, 96%, 97%, 98%, or 99% of the LCDR3-containing polypeptides is eluted from the chromatography column are unmodified.

In some embodiments, at least 50%, 55%, 60%, 65%, 70%, 75%, 80%, 85%, 86%, 87%, 88%, 89%, 90%, 91%, 92%, 93%, 94%, 95%, 96%, 97%, 98%, 99, or 100% of both the HCDR3-containing polypeptides and LCDR3-containing polypeptides eluted from the chromatography column are unmodified.

Various parameters can be modified in chromatography methods described herein. Parameters include protein loading, temperature of operation, conductivity of the protein being loaded onto the column, bed heights (chromatography column height), linear velocities, and pH. By way of example, protein loading can be at about 10 to about 200 g/L, 50 to about 200 g/L, about 55 to about 85 g/L, about 60 to about 80 g/L, about 65 to about 75 g/L, about 100 to about 200 g/L, about 100 to about 150 g/L, about 125 to about 175 g/L, about 150 to about 200 g/L, or about 90 to about 140 g/L. Temperature of operation on-column can be about 15° C. to about 25° C., or about 18° C. to about 22° C. The chromatography column height can be about 10 cm to about 35 cm, or about 20 cm to about 30 cm, or about 23 cm to about 27 cm (e.g., about 10 cm, about 11 cm, about 12 cm, about 13 cm, about 14 cm, about 15 cm, about 16 cm, about 17 cm, about 18 cm, about 19 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, or about 35 cm in height, including ranges between any two of the listed values). The linear velocity can be about 10 cm/hr to about 250 cm/hr, or about 120 cm/hr to about 220 cm/hr, about 125 cm/hr to about 165 cm/hr, or about 180 cm/hr to about 210 cm/hr. The pH can be about 5 to about 9, about 5 to about 7, about 7 to about 9, about 6 to about 8, about 5.5 to about 8.5, about 6.5 to about 8.5, about 5 to about 6, about 8 to about 9, about 7 to about 8, about 7 to about 7.5, or about 7.5 to about 8. In various cases, the pH is ±2 pH units of the pI of the protein of interest, or ±1 pH unit of the pI of the protein of interest, or ±0.5 pH units of the pI of the protein of interest. A sample solution and/or a formulation used in the chromatography may have a pH as described herein. The conductivity of the protein being loaded onto the column can be about 10 to about 50 mS/cm, about 10 to about 20 mS/cm, about 15 to about 25 mS/cm, about 10 to about 30 mS/cm, about 10 to about 40 mS/cm, about 20 to about 50 mS/cm, about 30 to about 50 mS/cm, about 40 to about 50 mS/cm, about 20 to about 30 mS/cm, about 30 to about 40 mS/cm, or about 15 to about 30 mS/cm.

The length of the exposure time of mobile phase B to the chromatography column is at least 15 minutes. In some embodiments, the length of exposure time of mobile phase B to the chromatography column ranges from 15 minutes to 3 hours, to 6 hours, to 12 hours, or to 24 hours, In some embodiments, the length of exposure time of wash to column is about 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 49 minutes, 50 minutes, 51 minutes, 52 minutes, 53 minutes, 54 minutes, 55 minutes, 56 minutes, 57 minutes, 58 minutes, 59 minutes, 1 hour, 2 hours, 3 hours, 4 hours, 5 hours, 6 hours, 7 hours, 8 hours, 9 hours, 10 hours, 11 hours 12 hours, 13 hours, 14 hours, 15 hours, 16 hours, 17 hours, 18 hours, 19 hours, 20 hours, 21 hours, 22 hours, 23 hours, 24 hours or longer, including ranges between any two of the listed values. In some embodiments, peptide mapping is performed in about 5 hours or less, for example 2-3 hours, 2-4 hours, 2-5 hours, 3-4 hours, or 3-5 hours.

Spectrometric Methods

In some embodiments, the methods described herein further comprise the step of performing spectrometric analysis of the eluted polypeptides. Exemplary methods for spectrometric analysis include, but are not limited to, mass spectrometry (Rubakhin and Sweedler, 2010), ultraviolet spectrometry, visible light spectrometry, fluorescent spectrometry, ultraviolet-visible light spectrometry, and infrared spectrometry.

The principle underlying mass spectrometry (MS) includes ionizing chemical compounds to generate charged molecules or molecule fragments, and then measuring their mass-to-charge ratios. In an illustrative MS procedure, a sample is loaded onto the MS instrument and undergoes vaporization, the components of the sample are ionized by one of a variety of methods (e.g., by impacting them with an electron beam), which results in the formation of positively charged particles, the positive ions are then accelerated by a magnetic field, computations are performed on the mass-to-charge ratio (m/z) of the particles based on the details of motion of the ions as they transit through electromagnetic fields, and, detection of the ions, which have been sorted according to their m/z ratios.

An illustrative MS instrument has three modules: an ion source, which converts gas phase sample molecules into ions (or, in the case of electrospray ionization, move ions that exist in solution into the gas phase); a mass analyzer, which sorts the ions by their mass-to-charge ratios by applying electromagnetic fields; and a detector, which measures the value of an indicator quantity and thus provides data for calculating the abundances of each ion present.

The MS technique has both qualitative and quantitative uses, including identifying unknown compounds, determining the isotopic composition of elements in a molecule, and determining the structure of a compound by observing its fragmentation. Examples include gas chromatography-mass spectrometry (GC/MS or GC-MS), liquid chromatography mass spectrometry (LC/MS or LC-MS), ion mobility spectrometry/mass spectrometry (IMS/MS or IMMS), matrix-assisted laser desorption/ionization source configured with a TOF analyzer (MALDI-TOF); electrospray ionization-mass spectrometry (ESI-MS), inductively coupled plasma-mass spectrometry (ICP-MS), accelerator mass spectrometry (AMS), thermal ionization-mass spectrometry (TIMS), and spark source mass spectrometry (SSMS).

Therapeutic Proteins

In some embodiments, the protein processed in any of the methods described herein is a therapeutic protein. In exemplary aspects, the therapeutic protein is an antibody. As used herein, the term “antibody” refers to a protein having a conventional immunoglobulin format, comprising heavy and light chains, and comprising variable and constant regions. For example, an antibody can be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be fragmented into fragments by enzymes, such as, e.g., papain and pepsin. Papain cleaves an antibody to produce two Fab fragments and a single Fc fragment. Pepsin cleaves an antibody to produce a F(ab′)2 fragment and a pFc′ fragment. In exemplary aspects of the present disclosure, the therapeutic protein is an antigen binding fragment or an antibody. As used herein, the term “antigen binding antibody fragment” refers to a portion of an antibody that is capable of binding to the antigen of the antibody and is also known as “antigen-binding fragment” or “antigen-binding portion”. In exemplary instances, the antigen binding antibody fragment is a Fab fragment or a F(ab′)₂ fragment.

In various aspects, the therapeutic protein is an antibody protein product. As used herein, the term “antibody protein product” refers to any one of several antibody alternatives which in various instances is based on the architecture of an antibody but is not found in nature. In some aspects, the antibody protein product has a molecular-weight within the range of at least about 12-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the therapeutic protein is a bispecific T cell engager (BiTE®) molecule, which is an artificial bispecific monoclonal antibody. Canonical BiTE® molecules are fusion proteins comprising two scFvs of different antibodies. One binds to CD3 and the other binds to a target antigen. BiTE® molecules are known in the art. See, e.g., Huehls et al., Immuno Cell Biol 93(3): 290-296 (2015); Rossi et al., MAbs 6(2): 381-91 (2014); Ross et al., PLoS One 12(8): e0183390.

In exemplary aspects, the therapeutic protein is a chimeric antigen receptor (CAR). Chimeric antigen receptors are genetically engineered fusion proteins constructed from multiple domains typically of other naturally occurring molecules expressed by immune cells. In several aspects, CARs comprises an extracellular antigen-binding domain or antigen recognition domain, a signaling domain and a co-stimulatory domain. CARs are described in the art. See, e.g., Maus et al., Clin Cancer Res 22(8): 1875-1884 (2016); Dotti et al., Immuno Rev (2014) 257(1): 10.1111/imr.12131; Lee et al., Clin Cancer Res (2012): 18(10): 2780-2790; and June and Sadelain, NEJM 379: 64-73 (2018).

Exemplary therapeutic proteins include but are not limited to, CD proteins, growth factors, growth factor receptor proteins (e.g., HER receptor family proteins), cell adhesion molecules (for example, LFA-I, MoI, p150, 95, VLA-4, ICAM-I, VCAM, and alpha v/beta 3 integrin), hormone (e.g., insulin), coagulation factors, coagulation-related proteins, colony stimulating factors and receptors thereof, and other receptors and receptor-associated proteins or ligands of these receptors, viral antigens.

Exemplary therapeutic proteins include, e.g., any one of the CD proteins, such as CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31,CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79α, CD79β, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD 115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

Exemplary growth factors, include, for instance, vascular endothelial growth factor (“VEGF”), growth hormone, thyroid stimulating hormone (TSH), follicle stimulating hormone (FSH), luteinizing hormone (LH), growth hormone releasing factor (GHRF), parathyroid hormone (PTH), Mullerian-inhibiting substance (MIS), human macrophage inflammatory protein (MIP-I-alpha), erythropoietin (EPO), nerve growth factor (NGF), such as NGF-beta, platelet-derived growth factor (PDGF), fibroblast growth factors (FGF), including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors. The therapeutic protein in some aspects is an insulin or insulin-related protein, e.g., insulin, insulin A-chain, insulin B-chain, proinsulin, and insulin-like growth factor binding proteins. Exemplary growth factor receptors include any receptor of any of the above growth factors. In various aspects, the growth factor receptor is a HER receptor family protein (for example, HER2, HER3, HER4, and the EGF receptor), a VEGF receptor, TSH receptor, FSH receptor, LH receptor, GHRF receptor, PTH receptor, MIS receptor, MIP-1-alpha receptor, EPO receptor, NGF receptor, PDGF receptor, FGF receptor, EGF receptor, (EGFR), TGF receptor, or insulin receptor.

Exemplary coagulation and coagulation-related proteins, include, for instance, factor VIII, tissue factor, von Willebrands factor, protein C, alpha-1-antitrypsin, plasminogen activators, such as urokinase and tissue plasminogen activator (“t-PA”), bombazine, thrombin, and thrombopoietin; (vii) other blood and serum proteins, including but not limited to albumin, IgE, and blood group antigens. Colony stimulating factors and receptors thereof, including the following, among others, M-CSF, GM-CSF, and G-CSF, and receptors thereof, such as CSF-1 receptor (c-fms). Receptors and receptor-associated proteins, including, for example, flk2/flt3 receptor, obesity (OB) receptor, LDL receptor, growth hormone receptors, thrombopoietin receptors (“TPO-R,” “c-mpl”), glucagon receptors, interleukin receptors, interferon receptors, T-cell receptors, stem cell factor receptors, such as c-Kit, and other receptors. Receptor ligands, including, for example, OX40L, the ligand for the OX40 receptor. Neurotrophic factors, including bone-derived neurotrophic factor (BDNF) and neurotrophin-3, -4, -5, or -6 (NT-3, NT-4, NT-5, or NT-6). Relaxin A-chain, relaxin B-chain, and prorelaxin; interferons and interferon receptors, including for example, interferon-α, -β, and -γ, and their receptors. Interleukins and interleukin receptors, including IL-I to IL-33 and IL-I to IL-33 receptors, such as the IL-8 receptor, among others. Viral antigens, including an HIV envelope viral antigen. Lipoproteins, calcitonin, glucagon, atrial natriuretic factor, lung surfactant, tumor necrosis factor-alpha and -beta, enkephalinase, RANTES (regulated on activation normally T-cell expressed and secreted), mouse gonadotropin-associated peptide, DNAse, inhibin, and activin. Integrin, protein A or D, rheumatoid factors, immunotoxins, bone morphogenetic protein (BMP), superoxide dismutase, surface membrane proteins, decay accelerating factor (DAF), HIV envelope, transport proteins, homing receptors, addressins, regulatory proteins, immunoadhesins, antibodies. Additional exemplary therapeutic proteins include, e.g., myostatins, TALL proteins, including TALL-I, amyloid proteins, including but not limited to amyloid-beta proteins, thymic stromal lymphopoietins (“TSLP”), RANK ligand (RANKL or “OPGL”), c-kit, TNF receptors, including TNF Receptor Type 1, TRAIL-R2, angiopoietins, and biologically active fragments or analogs or variants of any of the foregoing.

In exemplary aspects, the therapeutic protein is any one of the pharmaceutical agents known as Activase® (Alteplase); alirocumab, Aranesp® (Darbepoetin-alfa), Epogen® (Epoetin alfa, or erythropoietin); Avonex® (Interferon β-Ia); Bexxar® (Tositumomab); Betaseron® (Interferon-β); bococizumab (anti-PCSK9 monoclonal antibody designated as L1L3, see U.S. Pat. No. 8,080,243); Campath® (Alemtuzumab); Dynepo® (Epoetin delta); Velcade® (bortezomib); MLN0002 (anti-α4β7 mAb); MLN1202 (anti-CCR2 chemokine receptor mAb); Enbrel® (etanercept); Eprex® (Epoetin alfa); Erbitux® (Cetuximab); evolocumab; Genotropin® (Somatropin); Herceptin® (Trastuzumab); Humatrope® (somatropin [rDNA origin] for injection); Humira® (Adalimumab); Infergen® (Interferon Alfacon-1); Natrecor® (nesiritide); Kineret® (Anakinra), Leukine® (Sargamostim); LymphoCide® (Epratuzumab); Benlysta™ (Belimumab); Metalyse® (Tenecteplase); Mircera® (methoxy polyethylene glycol-epoetin beta); Mylotarg® (Gemtuzumab ozogamicin); Raptiva® (efalizumab); Cimzia® (certolizumab pegol); Soliris™ (Eculizumab); Pexelizumab (Anti-C5 Complement); MEDI-524 (Numax®); Lucentis® (Ranibizumab); Edrecolomab (Panorex®); Trabio® (lerdelimumab); TheraCim hR3 (Nimotuzumab); Omnitarg (Pertuzumab, 2C4); Osidem® (IDM-I); OvaRex® (B43.13); Nuvion® (visilizumab); Cantuzumab mertansine (huC242-DM1); NeoRecormon® (Epoetin beta); Neumega® (Oprelvekin); Neulasta® (Pegylated filgastrim, pegylated G-CSF, pegylated hu-Met-G-CSF); Neupogen® (Filgrastim); Orthoclone OKT3® (Muromonab-CD3), Procrit® (Epoetin alfa); Remicade® (Infliximab), Reopro® (Abciximab), Actemra® (anti-IL6 Receptor mAb), Avastin® (Bevacizumab), HuMax-CD4 (zanolimumab), Rituxan® (Rituximab); Tarceva® (Erlotinib); Roferon-A®-(Interferon alfa-2a); Simulect® (Basiliximab); Stelara™ (Ustekinumab); Prexige® (lumiracoxib); Synagis® (Palivizumab); 146B7-CHO (anti-IL15 antibody, see U.S. Pat. No. 7,153,507), Tysabri® (Natalizumab); Valortim® (MDX-1303, anti-B. anthracis Protective Antigen mAb); ABthrax™; Vectibix® (Panitumumab); Xolair® (Omalizumab), ETI211 (anti-MRSA mAb), IL-I Trap (the Fc portion of human IgG1 and the extracellular domains of both IL-I receptor components (the Type I receptor and receptor accessory protein)), VEGF Trap (Ig domains of VEGFR1 fused to IgG1 Fc), Zenapax® (Daclizumab); Zenapax® (Daclizumab), Zevalin® (Ibritumomab tiuxetan), Zetia (ezetimibe), Atacicept (TACI-Ig), anti-α4β7 mAb (vedolizumab); galiximab (anti-CD80 monoclonal antibody), anti-CD23 mAb (lumiliximab); BR2-Fc (huBR3/huFc fusion protein, soluble BAFF antagonist); Simponi™ (Golimumab); Mapatumumab (human anti-TRAIL Receptor-1 mAb); Ocrelizumab (anti-CD20 human mAb); HuMax-EGFR (zalutumumab); M200 (Volociximab, anti-α5β1 integrin mAb); MDX-010 (Ipilimumab, anti-CTLA-4 mAb and VEGFR-I (IMC-18F1); anti-BR3 mAb; anti-C. difficile Toxin A and Toxin B C mAbs MDX-066 (CDA-I) and MDX-1388); anti-CD22 dsFv-PE38 conjugates (CAT-3888 and CAT-8015); anti-CD25 mAb (HuMax-TAC); anti-TSLP antibodies; anti-TSLP receptor antibody (U.S. Pat. No. 8,101,182); anti-TSLP antibody designated as A5 (U.S. Pat. No. 7,982,016); (anti-CD3 mAb (NI-0401); Adecatumumab (MT201, anti-EpCAM-CD326 mAb); MDX-060, SGN-30, SGN-35 (anti-CD30 mAbs); MDX-1333 (anti-IFNAR); HuMax CD38 (anti-CD38 mAb); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-sclerostin antibodies (see, U.S. Pat. No. 8,715,663 or U.S. Pat. No. 7,592,429) anti-sclerostin antibody designated as Ab-5 (U.S. Pat. No. 8,715,663 or U.S. Pat. No. 7,592,429); anti-ganglioside GM2 mAb; anti-GDF-8 human mAb (MYO-029); anti-GM-CSF Receptor mAb (CAM-3001); anti-HepC mAb (HuMax HepC); MEDI-545, MDX-1103 (anti-IFNα mAb); anti-IGFIR mAb; anti-IGF-IR mAb (HuMax-Inflam); anti-IL12/IL23p40 mAb (Briakinumab); anti-IL-23p19 mAb (LY2525623); anti-IL13 mAb (CAT-354); anti-IL-17 mAb (AIN457); anti-IL2Ra mAb (HuMax-TAC); anti-IL5 Receptor mAb; anti-integrin receptors mAb (MDX-018, CNTO 95); anti-IPIO Ulcerative Colitis mAb (MDX-1100); anti-LLY antibody; BMS-66513; anti-Mannose Receptor/hCGβ mAb (MDX-1307); anti-mesothelin dsFv-PE38 conjugate (CAT-5001); anti-PD1mAb (MDX-1 106 (ONO-4538)); anti-PDGFRα antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; anti-ZP3 mAb (HuMax-ZP3); NVS Antibody #1; NVS Antibody #2; or an amyloid-beta monoclonal antibody.

Additional examples of therapeutic proteins include antibodies such as infliximab, bevacizumab, cetuximab, ranibizumab, palivizumab, abagovomab, abciximab, actoxumab, adalimumab, afelimomab, afutuzumab, alacizumab, alacizumab pegol, ald518, alemtuzumab, alirocumab, altumomab, amatuximab, anatumomab mafenatox, anrukinzumab, apolizumab, arcitumomab, aselizumab, altinumab, atlizumab, atorolimiumab, tocilizumab, bapineuzumab, basiliximab, bavituximab, bectumomab, belimumab, benralizumab, bertilimumab, besilesomab, bevacizumab, bezlotoxumab, biciromab, bivatuzumab, bivatuzumab mertansine, blinatumomab, blosozumab, brentuximab vedotin, briakinumab, brodalumab, canakinumab, cantuzumab mertansine, cantuzumab mertansine, caplacizumab, capromab pendetide, carlumab, catumaxomab, cc49, cedelizumab, certolizumab pegol, cetuximab, citatuzumab bogatox, cixutumumab, clazakizumab, clenoliximab, clivatuzumab tetraxetan, conatumumab, crenezumab, cr6261, dacetuzumab, daclizumab, dalotuzumab, daratumumab, demcizumab, denosumab, detumomab, dorlimomab aritox, drozitumab, duligotumab, dupilumab, ecromeximab, eculizumab, edobacomab, edrecolomab, efalizumab, efungumab, elotuzumab, elsilimomab, enavatuzumab, enlimomab pegol, enokizumab, enoticumab, ensituximab, epitumomab cituxetan, epratuzumab, erenumab, erlizumab, ertumaxomab, etaracizumab, etrolizumab, evolocumab, exbivirumab, fanolesomab, faralimomab, farletuzumab, fasinumab, fbta05, felvizumab, fezakinumab, ficlatuzumab, figitumumab, flanvotumab, fontolizumab, foralumab, foravirumab, fresolimumab, fulranumab, futuximab, galiximab, ganitumab, gantenerumab, gavilimomab, gemtuzumab ozogamicin, gevokizumab, girentuximab, glembatumumab vedotin, golimumab, gomiliximab, gs6624, ibalizumab, ibritumomab tiuxetan, icrucumab, igovomab, imciromab, imgatuzumab, inclacumab, indatuximab ravtansine, infliximab, intetumumab, inolimomab, inotuzumab ozogamicin, ipilimumab, iratumumab, itolizumab, ixekizumab, keliximab, labetuzumab, lebrikizumab, lemalesomab, lerdelimumab, lexatumumab, libivirumab, ligelizumab, lintuzumab, lirilumab, lorvotuzumab mertansine, lucatumumab, lumiliximab, mapatumumab, maslimomab, mavrilimumab, matuzumab, mepolizumab, metelimumab, milatuzumab, minretumomab, mitumomab, mogamulizumab, morolimumab, motavizumab, moxetumomab pasudotox, muromonab-cd3, nacolomab tafenatox, namilumab, naptumomab estafenatox, narnatumab, natalizumab, nebacumab, necitumumab, nerelimomab, nesvacumab, nimotuzumab, nivolumab, nofetumomab merpentan, ocaratuzumab, ocrelizumab, odulimomab, ofatumumab, olaratumab, olokizumab, omalizumab, onartuzumab, oportuzumab monatox, oregovomab, orticumab, otelixizumab, oxelumab, ozanezumab, ozoralizumab, pagibaximab, palivizumab, panitumumab, panobacumab, parsatuzumab, pascolizumab, pateclizumab, patritumab, pemtumomab, perakizumab, pertuzumab, pexelizumab, pidilizumab, pintumomab, placulumab, ponezumab, priliximab, pritumumab, PRO 140, quilizumab, racotumomab, radretumab, rafivirumab, ramucirumab, ranibizumab, raxibacumab, rontalizumab, rovelizumab, ruplizumab, samalizumab, sarilumab, satumomab pendetide, secukinumab, sevirumab, sibrotuzumab, sifalimumab, siltuximab, simtuzumab, siplizumab, sirukumab, solanezumab, solitomab, sonepcizumab, sontuzumab, stamulumab, sulesomab, suvizumab, tabalumab, tacatuzumab tetraxetan, tadocizumab, talizumab, tanezumab, taplitumomab paptox, tefibazumab, telimomab aritox, tenatumomab, tefibazumab, teneliximab, teplizumab, teprotumumab, tezepelumab, TGN1412, tremelimumab, ticilimumab, tildrakizumab, tigatuzumab, TNX-650, tocilizumab, toralizumab, tositumomab, tralokinumab, trastuzumab, TRBS07, tregalizumab, tucotuzumab celmoleukin, tuvirumab, ublituximab, urelumab, urtoxazumab, ustekinumab, vapaliximab, vatelizumab, vedolizumab, veltuzumab, vepalimomab, vesencumab, visilizumab, volociximab, vorsetuzumab mafodotin, votumumab, zalutumumab, zanolimumab, zatuximab, ziralimumab, and zolimomab aritox.

TABLE A
Examples of therapeutic antibodies

| Target | HC* Type | | | LC* | HC* |
|---|---|---|---|---|---|
| (informal | (including | LC* | | SEQ ID | SEQ ID |
| name) | allotypes) | Type | pI | NO: | NO: |
| anti- | IgG1 (f) (R; EM) | Kappa | 9.0 | 1 | 2 |
| amyloid | | | | | |
| GMCSF | IgG2 | Kappa | 8.7 | 3 | 4 |
| (247) | | | | | |
| CGRPR | IgG2 | Lambda | 8.6 | 5 | 6 |
| RANKL | IgG2 | Kappa | 8.6 | 7 | 8 |
| Sclerostin | IgG2 | Kappa | 6.6 | 9 | 10 |
| (27H6) | | | | | |
| IL-1R1 | IgG2 | Kappa | 7.4 | 11 | 12 |
| Myostatin | IgG1 (z) (K; EM) | Kappa | 8.7 | 13 | 14 |
| B7RP1 | IgG2 | Kappa | 7.7 | 15 | 16 |
| Amyloid | IgG1 (za) (K; DL) | Kappa | 8.7 | 17 | 18 |
| GMCSF | IgG2 | Kappa | 8.8 | 19 | 20 |
| (3.112) | | | | | |
| CGRP | IgG2 | Kappa | 8.7 | 21 | 22 |
| (32H7) | | | | | |
| CGRP | IgG2 | Lambda | 8.6 | 23 | 24 |
| (3B6.2) | | | | | |
| PCSK9 | IgG2 | Kappa | 6.7 | 25 | 26 |
| (8A3.1) | | | | | |
| PCSK9 | IgG2 | Kappa | 6.9 | 27 | 28 |
| (492) | | | | | |
| CGRP | IgG2 | Lambda | 8.8 | 29 | 30 |
| Hepcidin | IgG2 | Lambda | 7.3 | 31 | 32 |
| TNFR p55) | IgG2 | Kappa | 8.2 | 33 | 34 |
| OX40L | IgG2 | Kappa | 8.7 | 35 | 36 |
| HGF | IgG2 | Kappa | 8.1 | 37 | 38 |
| GMCSF | IgG2 | Kappa | 8.1 | 39 | 40 |
| Glucagon | IgG2 | Kappa | 8.4 | 41 | 42 |
| R | | | | | |
| GMCSF | IgG2 | Kappa | 8.4 | 43 | 44 |
| (4.381) | | | | | |
| Sclerostin | IgG2 | Kappa | 7.8 | 45 | 46 |
| (13F3) | | | | | |
| CD-22 | IgG1 (f) (R; EM) | Kappa | 8.8 | 47 | 48 |
| INFgR | IgG1 (za) (K; DL) | Kappa | 8.8 | 49 | 50 |
| Ang2 | IgG2 | Kappa | 7.4 | 51 | 52 |
| TRAILR2 | IgG1 (f) (R; EM) | Kappa | 8.7 | 53 | 54 |
| EGFR | IgG2 | Kappa | 6.8 | 55 | 56 |
| IL-4R | IgG2 | Kappa | 8.6 | 57 | 58 |
| IL-15 | IgG1 (f) (R; EM) | Kappa | 8.8 | 59 | 60 |
| IGF1R | IgG1 (za) (K; DL) | Kappa | 8.6 | 61 | 62 |
| IL-17R | IgG2 | Kappa | 8.6 | 63 | 64 |
| Dkk1 | IgG2 | Kappa | 8.2 | 65 | 66 |
| (6.37.5) | | | | | |
| Sclerostin | IgG2 | Kappa | 7.4 | 67 | 68 |
| TSLP | IgG2 | Lambda | 7.2 | 69 | 70 |
| Dkk1 | IgG2 | Kappa | 8.2 | 71 | 72 |
| (11H10) | | | | | |
| PCSK9 | IgG2 | Lambda | 8.1 | 73 | 74 |
| GIPR | IgG1 (z) (K; EM) | Kappa | 8.1 | 75 | 76 |
| (2G10.006) | | | | | |
| Activin | IgG2 | Lambda | 7.0 | 77 | 78 |
| Sclerostin | IgG2 | Lambda | 6.7 | 79 | 80 |
| (2B8) | | | | | |
| Sclerostin | IgG2 | Kappa | 6.8 | 81 | 82 |
| c-fms | IgG2 | Kappa | 6.6 | 83 | 84 |
| α4β7 | IgG2 | Kappa | 6.5 | 85 | 86 |
| PD-1 | IgG2 | Kappa | — | 87 | 88 |

*HC—antibody heavy chain; LC—antibody light chain.

In some embodiments, the therapeutic polypeptide is a BiTE® molecule. Blinatumomab (BLINCYTO®) is an example of a BiTE® molecule, specific for CD19. BiTE® molecules that are modified, such as those modified to extend their half-lives, can also be used in the disclosed methods.

All patents and other publications identified are expressly incorporated herein by reference in their entirety or in relevant part, as would be apparent from the context of the citation, for the purpose of describing and disclosing, for example, the methodologies described in such publications that might be used in connection with information described herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

The following examples are given merely to illustrate the present invention and not in any way to limit its scope.

### EXAMPLES

### Example 1—Conventional Peptide Mapping Under Reduced Conditions

Samples (100-500 mg) of monoclonal antibodies mAb A, mAb B, mAb C, and mAb D were denatured by diluting in denaturing buffer containing 0.25 M Tris, 7.5 M guanidine-HCl, pH 7.5 followed under reducing conditions, incubating in 0.5 M dithiothreitol (DTT) at room temperature for 25 minutes. Reduced samples were then alkylated using 0.5 M sodium iodoacetate/acetic acid and incubated in dark at room temperature for 20 min. The reduced, alkylated samples were then buffer-exchanged into digestion buffer (0.1 M Tris, pH 7.5) using size exclusion columns to remove the earlier buffer components. Next, samples were digested using trypsin endopeptidase at a ratio of 1:10 (enzyme:sample) and incubating at 37° C. for 30 minutes. The reaction was quenched by addition of trifluoroacetic acid to a final concentration of 1% (v/v). The digested samples are analyzed by liquid chromatography tandem-mass spectrometry (MS/MS).

The liquid chromatography MS/MS system consisted of an UPLC/HPLC system connected in-line to a mass spectrometer. Separation was achieved by injecting samples (10-50 ug) onto a C18/C8 stationary phase column kept at 50° C. and applying a linear gradient of 0%-40% mobile phase B, using 0.1% trifluoroacetic acid in water and 0.1% trifluoroacetic acid in acetonitrile as mobile phase A & B respectively, over a period of 210 min at flow rate of 0.1 mL/min. Data acquisition was performed in positive mode and each peptide was subjected to MS/MS for sequence information. The presence of post-translational modifications (PTMs) of the HCDR3- and LCDR3-containing polypeptides was assessed. Modification percentages were calculated by dividing the total area of the oxidized peptide by the sum of total areas from the oxidized and unoxidized peptides. The results as shown below in Table 1.

TABLE 1

| | No. of MS | | Sequence | Abundance |
|---|---|---|---|---|
| | Peaks | MS Peak Area | coverage | (mol) |
| mAb A | | | | |
| Light chain | 120 | 23.3% | 84.1% | 50.64% |
| Heavy chain | 278 | 43.3% | 91.5% | 49.36% |
| Unidentified | 3123 | 33.33% | | |
| mAb B | | | | |
| Light chain | 84 | 23.4% | 96.7% | 44.21% |
| Heavy chain | 187 | 47.1% | 81.6% | 55.79% |
| Unidentified | 964 | 29.5% | | |
| mAb C | | | | |
| Light chain | 72 | 20.1% | 75.7% | 40.72% |
| Heavy chain | 199 | 52.2% | 85.0% | 59.28% |
| Unidentified | 792 | 27.8% | | |
| mAb D | | | | |
| Light chain | 93 | 21.7% | 97.2% | 43.21% |
| Heavy chain | 217 | 54.0% | 93.1% | 56.79% |
| Unidentified | 727 | 24.3% | | |

Results showed that 84.1% of the unmodified (e.g., lack of PTMs) light chain and 91.5% of the unmodified heavy chain of mAb A were recovered in the eluant; 96.7% of the unmodified light chain and 81.6% of the unmodified heavy chain of mAb B were recovered in the eluant; 75.7% of the unmodified light chain and 85.0% of the unmodified heavy chain of mAb C were recovered in the eluant; and 97.2% of the unmodified light chain and 93.1% of the unmodified heavy chain of mAb D were recovered in the eluant. While a tryptophan in HCDR3 of mAb A had been identified as a sensitive site for oxidation, peptide containing this tryptophan was not recovered at all by the conventional method.

### Example 2—Conventional Peptide Mapping Under Non-Reduced Conditions

Samples (100-500 mg) mAb A were denatured using RapiGest (Waters Corp., Milford, MA) and then digested using endopeptidase trypsin under denaturing conditions in presence of NEM (n-ethyl maleimide) for overnight. The reaction was quenched by addition of trifluoroacetic acid to a final concentration of 1% (v/v). The digested samples were then analyzed by liquid chromatography tandem-mass spectrometry (MS/MS).

The liquid chromatography MS/MS system consisted of an UPLC/HPLC system connected in-line to a mass spectrometer. Separation was achieved by injecting samples (10-ug) onto a Waters Acquity BEH C4 stationary phase column kept at 50° C. and applying a linear gradient of 0%-40% mobile phase B, using 0.1% trifluoroacetic acid in water and trifluoroacetic acid in acetonitrile as mobile phase A & B respectively, over a period of 220 min at flow rate of 0.1 mL/min. Data acquisition was performed in positive mode and each peptide was subjected to MS/MS. The presence of post-translational modifications (PTMs) of the HCDR3- and LCDR3-containing polypeptides was assessed. Using sequence information, disulfide bond linkages were then determined using informatic tools and confirmed. The results are shown below in Table 2.

TABLE 2

| | No. of MS | | Sequence | Abundance |
|---|---|---|---|---|
| mAb A | Peaks | MS Peak Area | coverage | (mol) |
| Light chain | 158 | 22.9% | 95.5% | 51.54% |
| Heavy chain | 342 | 44.5% | 94.8% | 48.46% |
| Unidentified | 2870 | 32.6% | | |

Results showed that 95.5% of the unmodified light chain and 94.8% of the unmodified heavy chain were recovered in the eluent.

### Example 3—Higher Yield Peptide Mapping Method Under Reducing Conditions

Samples (100-500 mg) of mAb A, mAb B, mAb C or mAb D were denatured by diluting into denaturing buffer containing 0.25 M Tris, 7.5 M guanidine-HCl, 0.25 mM EDTA pH 7.5 followed by reduction by incubating in 0.5 M dithiothreitol (DTT) at RT for 25 minutes. Reduced samples were then alkylated using 0.5 M sodium iodoacetate/acetic acid and incubated in the dark at room temperature for 20 min. Subsequently, the reduced, alkylated samples were buffer-exchanged into digestion buffer (0.1 M Tris, pH 7.5) using size exclusion columns to remove the earlier buffer components. Next, samples were digested using trypsin endopeptidase using a ratio of 1:10 (enzyme:sample) and incubating at 37° C. for minutes. The reaction was quenched by addition of trifluoroacetic acid to a final concentration of 1% (v/v). The digested samples were then analyzed by liquid chromatography tandem-mass spectrometry (MS/MS).

The liquid chromatography MS/MS system consisted of an UPLC/HPLC system connected in-line to a mass spectrometer. Separation was achieved by injecting samples (10-ug) onto Agilent PLRP-S column kept at 50° C. and applying a linear gradient using water with 0.05-0.1% formic acid or 0.05-0.1% trifluoroacetic acid & 40% isopropyl alcohol/40% Acetonitrile/20% water with 0.05-0.1% formic acid or 0.05-0.1% trifluoroacetic acid going from 0%-48% as mobile phase B, over a period of 104 minutes at flow rate of 0.2 mL/min. Data acquisition was performed in positive mode and each peptide is subjected to MS/MS for sequence information. The presence of post-translational modifications (PTMs) of the HCDR3- and LCDR3-containing polypeptides was assessed. Modification percentages were calculated by dividing the total area of the oxidized peptide by the sum of total areas from the oxidized and unoxidized peptides. The results are shown below in Table 3.

TABLE 3

| | No. of MS | | Sequence | Abundance |
|---|---|---|---|---|
| | Peaks | MS Peak Area | coverage | (mol) |
| mAb A | | | | |
| Light chain | 144 | 23.4% | 100.0% | 48.05% |
| Heavy chain | 325 | 45.4% | 100.0% | 51.95% |
| Unidentified | 1920 | 31.2% | | |
| mAb B | | | | |
| Light chain | 87 | 15.5% | 96.3% | 43.92% |
| Heavy chain | 201 | 33.3% | 98.0% | 56.08% |
| Unidentified | 944 | 51.3% | | |
| mAb C | | | | |
| Light chain | 79 | 15.4% | 98.1% | 39.04% |
| Heavy chain | 214 | 41.9% | 96.6% | 60.96% |
| Unidentified | 877 | 42.7% | | |
| mAb D | | | | |
| Light chain | 80 | 13.3% | 98.1% | 40.21% |
| Heavy chain | 197 | 38.3% | 98.0% | 59.79% |
| Unidentified | 759 | 48.3% | | |

Results showed that 100% of both the unmodified light and heavy chains of mAb A were recovered in the eluant.

Results also showed that 96.3% of the unmodified (e.g., lack of PTMs) light chain and 98% of the unmodified heavy chain of mAb B were recovered in the eluant; 98.1% of the unmodified light chain and 96.6% of the unmodified heavy chain of mAb C were recovered in the eluant; and 98.1% of the unmodified light chain and 98.0% of the unmodified heavy chain of mAb D were recovered in the eluant.

### Example 4—Higher Yield Peptide Mapping Method Under Non-Reducing Conditions

Samples (100-500 mg), each containing a different HLE BITE® molecule (HLE-BiTE® A, HLE-BiTE® B, and HLE-BiTE® C) were denatured using RapiGest and then digested using endopeptidase trypsin under denaturing conditions in presence of NEM (n-ethyl maleimide) for overnight. The reaction was quenched by addition of trifluoroacetic acid to a final concentration of 1% (v/v). The digested samples were analyzed by liquid chromatography tandem-mass spectrometry (MS/MS).

The liquid chromatography MS/MS system consisted of an UPLC/HPLC system connected in-line to a mass spectrometer. Separation is achieved by injecting samples (10-50 ug) onto Agilent PLRP-S column, Acuity C8 column, or an Acuity C4 column kept at 50° C. and applying a linear gradient using 95% water/5% isopropyl alcohol with 0.1% formic acid & 40% isopropyl alcohol/40% Acetonitrile/20% water/0.1% formic acid going from 0%-35% B, over a period of 70 min at flow rate of 0.2 mL/min.

The peptide yield was also assessed under the conventional peptide mapping conditions. Briefly, samples (10-50 ug) were injected onto a Zorbax SB-C18, Acquity C18 column, or an Acquity C8 column kept at 50° C. and applying a linear gradient of 0%-40% mobile phase B, using 0.1% formic acid in water and 0.1% formic acid in acetonitrile as mobile phase A & B respectively, over a period of 220 min at flow rate of 0.1 mL/min.

Data acquisition was performed in positive mode and each peptide was subjected to MS/MS for sequence information. The presence of post-translational modifications (PTMs) of the HCDR3- and LCDR3-containing polypeptides was assessed. Using sequence information, disulfide bond linkages are then determined using informatic tools and confirmed.

Results, shown for HLE-BiTE® A in the Table 4 below, indicated that the method described in this Example using the PLRPS column and a mobile phase B comprising 40% isopropyl alcohol/40% Acetonitrile/20% water resulted in the identification of all peptides of interest (marked with an “X” in Table 4 below). Similar results were observed for HLE-BiTE® B and HLE-BiTE® C.

TABLE 4

| | Zorbax | Acquity | | | Acquity | Peptide |
|---|---|---|---|---|---|---|
| | SB-C18 | C18 | | | C8 | identified on |
| | (formic | (formic | Acquity C8 | Acquity C8 | (with | PLRPS column |
| | acid | acid | (formic acid | (with mobile | mobile | (with mobile |
| HLE-BiTE ® A | mobile | mobile | mobile | phase B | phase B | phase B |
| peptides | phases) | phases) | phases) | 40/40/20) | 40/40/20) | 40/40/20)_{—} |
| VH1 HCDR1 + | No ID | No ID | No ID | No ID | No ID | X |
| VH1 HCDR3 | | | | | | |
| VH1 HCDR2 + | X | X | X | X | X | X |
| VL1 LCDR3 | | | | | | |
| Non-CDR + | X | X | X | X | X | X |
| VL1 LCDR3 | | | | | | |
| Non-CDR + | X | X | X | X | X | X |
| VH2 HCDR1 | | | | | | |
| VH2 HCDR3 + | No ID | No ID | No ID | X | No ID | X |
| VL2 LCDR1 | | | | | | |
| VH2 HCDR3 + | No ID | No ID | No ID | X | No ID | X |
| VL2 LCDR1 + | | | | | | |
| VL2 LCDR3 | | | | | | |

*No ID = peptide was not identified

### Example 5—Head-to-Head Comparison of Columns with Performance Summary

Samples (100-500 mg) of mAb A were denatured by diluting into denaturing buffer containing 0.25 M Tris, 7.5 M guanidine-HCl, 0.25 mM EDTA pH 7.5 followed by reduction by incubating in 0.5 M dithiothreitol (DTT) at RT for 25 minutes. Reduced samples were then alkylated using 0.5 M sodium iodoacetate/acetic acid and incubated in the dark at room temperature for 20 min. Subsequently, the reduced, alkylated samples were buffer-exchanged into digestion buffer (0.1 M Tris, pH 7.5) using size exclusion columns to remove the earlier buffer components. Next, samples were digested using trypsin endopeptidase using a ratio of 1:10 (enzyme:sample) and incubating at 37° C. for 30 minutes. The reaction was quenched by addition of trifluoroacetic acid to a final concentration of 1% (v/v). The digested samples were then analyzed by liquid chromatography tandem-mass spectrometry (MS/MS).

The liquid chromatography MS/MS system consisted of an UPLC/HPLC system connected in-line to a mass spectrometer. Separation was achieved by one of the following methods:
(Method 1) injecting samples (10-50 ug) onto a Waters Acquity BEHC130 C18 stationary phase column kept at 50° C. and applying a linear gradient of 0%-40% mobile phase B, using 0.1% trifluoroacetic acid in water and 0.1% trifluoroacetic acid in acetonitrile as mobile phase A & B respectively, over a period of 220 min at flow rate of 0.1 mL/min;
(Method 2) injecting samples (10-50 ug) onto a Waters Acquity BEHC130 C18 stationary phase column kept at 50° C. and applying a linear gradient of 0%-48% mobile phase B, using 0.1% trifluoroacetic acid in water as mobile phase A and 0.05-0.1% trifluoroacetic acid & 40% isopropyl alcohol/40% Acetonitrile/20% water as mobile phase B, over a period of 220 min at flow rate of 0.1 mL/min; or a

(Method 3) injecting samples (10-50 ug) onto an Agilent PLRP-S column kept at 50° C. and applying a linear gradient using water with using 0.1% trifluoroacetic acid in water as mobile phase A and 0.05-0.1% trifluoroacetic acid & 40% isopropyl alcohol/40% Acetonitrile/20% water as mobile phase over a period of 220 min at flow rate of 0.1 mL/min.

Data acquisition was performed in positive mode and each peptide is subjected to MS/MS for sequence information. The presence of post-translational modifications (PTMs) of the HCDR3- and LCDR3-containing polypeptides was assessed. Modification percentages were calculated by dividing the total area of the oxidized peptide by the sum of total areas from the oxidized and unoxidized peptides. The results are shown below in Table 5.

TABLE 5

| Parameter | Method 1 | Method 2 | Method 3 |
|---|---|---|---|
| Recovery of HCDR3 | N/A | >10% | >50% |
| peptide | | | |
| % Sequence | 84.1% | 88% | 100% |
| coverage (LC) | | | |
| % Sequence | 91.5% | 95% | 100% |
| coverage (HC) | | | |
| Total number of | 33 | 37 | 44 |
| modifications | | | |
| Unique | — | 4 | 15 |
| modifications | | | |

Results showed that 100% of both the unmodified light and heavy chains of mAb A were recovered in the eluant under the conditions of Method 3. Thus, the data in this Example demonstrates that the PLRP-S column of Method 3 was superior to the C18 column of Method when subjected to the same conditions (100% v. 88%, respectively, for % LC sequence coverage; and 100% v. 95%, respectively, for % HC sequence overage). In addition, the use of a PLRP-S column in conjunction with the mobile phase B solvent comprising TFA, acetonitrile and alcohol was far superior to the conventional method (i.e., Method 1) for processing a protein.

### Example 6— Comparison of Additional Columns and Performance Summary

The method described in Example 3 was repeated using various columns under reducing conditions to compare HLE-BiTE® B peptide mapping performance. Columns assessed include PLRP-S, Polaris C18-Ether, Polaris C8-Ether, Peptide HSS T3, CORTECS T3, CORTECS C8, CORTECS phenyl and CSH C18. Results are shown below in Table 6.

TABLE 6

| | | Peptide recovery (hydrophobic | | | | |
|---|---|---|---|---|---|---|
| | | containing PTMs | | | | |
| | Sequence | Peptide 1 | | | Total # of | # of unique |
| Column | coverage | (HCDR3) | Peptide 2 | Peptide 3 | modifications | modifications |
| PLRP-S | 95.7% | >20% | >10% | >5% | 41 | 4 |
| Polaris | 96.10% | >10% | >10% | >5% | 44 | 3 |
| C18-Ether | | | | | | |
| Polaris C8- | 91.50% | >10% | >5% | >5% | 41 | 3 |
| Eher | | | | | | |
| Peptide | 95.6% | >10% | >5% | >5% | 47 | 5 |
| HSS T3 | | | | | | |
| CORTECS | 94.7% | >5% | >2% | >5% | 32 | 1 |
| T3 | | | | | | |
| CORTECS | 92.3% | >5% | >2% | >5% | 44 | 3 |
| C8 | | | | | | |
| CORTECS | 95.2% | >5% | >1% | >5% | 42 | 3 |
| phenyl | | | | | | |
| CSH C18 | 92.7% | >5% | >1% | >5% | 43 | 2 |

As shown in Table 5, a higher peptide of peptide 1 (HLE-BiTE® B CDR3) recovery (>20%) was observed when using the PLRP-S column. The >20% recovery permitted peptide mapping that reliably covered the residues of HLE-BiTE® B CDR3.

1. A method of processing a protein, the method comprising:
fragmenting the protein, thereby producing polypeptides;
applying the polypeptides to a chromatography column; and
eluting the polypeptides in an eluant comprising a mobile phase B solvent comprising:
trifluoroacetic acid (TFA);
acetonitrile; and
alcohol.

2. The method of claim 1, wherein the protein comprises and the polypeptides comprise a HCDR3 of a heavy chain variable region and/or a LCDR3 of a light chain variable region.

3. The method of claim 2, further comprising constructing a structural map of the protein, wherein the structural map comprises the HCDR3 and the LCDR3.

4. The method of claim 1, wherein the mobile phase B comprises 0.05%-0.09% TFA.

5. The method of claim 2, wherein at least 50% of the polypeptides that comprise the HCDR3 and/or LCDR3 are eluted from the chromatography column.

6. (canceled)

7. (canceled)

8. The method of claim 1, wherein said eluting is on a gradient of the mobile phase B solvent and a polar mobile phase A solvent.

9. The method of claim 8, wherein the mobile phase A comprises TFA and water.

10. (canceled)

11. The method of claim 1, wherein the chromatography column comprises porous particles having a particle size of about 2-5 μm, 2-7 μm, 3-5 μm, or 3-7 μm.

12. The method of claim 11, wherein the porous particles each have a pore size of about 100-500 angstroms.

13. The method of claim 1, wherein the chromatography column comprises fully porous particles having a pore size of about 300 angstroms and a particle size of about 5 μm.

14. The method of claim 11, wherein the chromatography column is at least 10 cm in height.

15. (canceled)

16. (canceled)

17. The method of claim 1, wherein the chromatography column comprises a divinylbenzene (DVB) resin.

18. The method of claim 1, wherein the protein is reduced.

19. (canceled)

20. The method of claim 1, further comprising analyzing the eluted polypeptides by spectrometric analysis.

21. The method of claim 1, wherein the protein comprises a therapeutic protein.

22. The method of claim 1, wherein the protein is selected from the group consisting of an antibody or antigen-binding fragment thereof, a derivative of an antibody or antibody fragment, and a fusion polypeptide.

23. The method of claim 1, wherein the protein is a bi-specific T-cell engager molecule.

24. (canceled)

25. A chromatography column comprising:
polypeptide fragments of a protein; and
an eluant comprising a mobile phase B solvent comprising:
trifluoroacetic acid (TFA);
acetonitrile; and
alcohol.

26. The chromatography column of claim 25, wherein the protein comprises a CDR of a variable region, such as a HCDR3 of a heavy chain variable region and/or a LCDR3 of a light chain variable region.

27.-36. (canceled)

37. The chromatography column of claim 25, comprising fully porous particles having a pore size of about 300 angstroms and a particle size of about 3 μm.

38.-42. (canceled)
